(19) United States
(12) Patent Application Publication
(10) Pub. No.: US 2021/0214663 A1
(43) Pub. Date: Jul. 15, 2021

(54) COLLECTIONS AND MEASUREMENTS OF MICROFLUIDIC SAMPLES

(71) Applicant: Hewlett-Packard Development Company, L.P., Spring, TX (US)

(72) Inventors: Steven Barcelo, Palo Alto, CA (US); Anita Rogacs, San Diego, CA (US); Fausto D'Apuzzo, Palo Alto, CA (US); Caitlin DeJong, Palo Alto, CA (US); Milo Overbay, Corvallis, OR (US)

(73) Assignee: Hewlett-Packard Development Company, L.P., Spring, TX (US)

(21) Appl. No.: 17/047,349

(22) PCT Filed: Aug. 20, 2018
(86) PCT No.: PCT/US2018/047107

§ 371 Date: Oct. 13, 2020

(51) Int. Cl. C12M 1/32; C12M 1/26; G01N 21/3577; C12Q 1/18; C12M 3/06

(52) U.S. Cl. CPC C12M 23/12; C12M 33/04; C12M 23/16; C12Q 1/18; G01N 21/3577

(57) ABSTRACT

An example of an apparatus includes a plurality of wells, wherein each well is to receive a cell culture and a cell culture liquid. The apparatus also includes a collector to contact the cell culture liquid in a well selected from the plurality of wells. The collector is to draw a microfluidic sample of the cell culture liquid from the well. In addition, the apparatus includes a sensor substrate to receive the microfluidic sample, wherein the sensor substrate is to be used to measure a characteristic of the cell culture liquid. Also, the apparatus includes a microfluidic channel to transport the microfluidic sample from the collector to the sensor substrate.

### BACKGROUND

Collecting samples of a cell culture for measurements may be used in various industries, such as biology and medicine. For example, large samples may include cells that may be counted or turbidity that may be measured to determine the density of cells in a given volume. This may provide an ability to make evaluations for several different applications. For example, measuring cells may have applications in antimicrobial susceptibility testing, such as for determining a minimum inhibitory concentration.

### BRIEF DESCRIPTION OF THE DRAWINGS

Reference will now be made, by way of example only, to the accompanying drawings in which:

FIG. 1 is a schematic diagram of an example apparatus to collect microfluidic samples;

FIG. 2a is a schematic diagram of another example apparatus to collect microfluidic samples and measure a characteristic;

FIG. 2b is a schematic diagram of zoomed in view of the example collector and microfluidic channel of FIG. 2a;

FIG. 3 is a schematic diagram of an example apparatus to collect microfluidic samples;

FIG. 4 is flowchart of an example method of collecting microfluidic samples; and

FIG. 5 is flowchart of an example method of collecting microfluidic samples to determine a minimum inhibitory concentration of an antibiotic.

### DETAILED DESCRIPTION

Measurements of the health of cells in a culture may have many applications and may involve many techniques. For example, an application of measuring cells may be to determine bacteria cell health during antimicrobial susceptibility testing, such as to determine a minimum inhibitory concentration of an antibiotic during a testing phase for an antibiotic. One method to determine the minimum inhibitory concentration involves dispensing antibiotics of varying concentration into separate wells containing bacteria. Each well may then be monitored, such as by observing the turbidity in the well. It is to be appreciated that by using this method, the minimum inhibitory concentration may be determined after sufficient time elapses that enough cells grow in a well for a reliable positive turbidity measurement, which may be about 24 to 48 hours. Accordingly, early detection or measurements may not be available. In other examples, rapid minimum inhibitory concentration determination may be made by directly measuring biomarkers indicating cell health using a spectroscopic technique such as surface-enhanced Raman spectroscopy or surface-enhanced Infrared absorption spectroscopy. However, this may involve collecting a sample size that disturbs the cell culture growth and introduces additional variables that affect the measurements.

The biomarkers that are measured are not particularly limited and may be specific to a type of cell culture or application. For example, metabolites from purine degradation such as adenine, uric acid and hypoxanthine may be used to identify bacteria and bacterial health when the cell culture is a bacteria culture. Tracking the concentration of such metabolites over time may provide an indication of the health of the cell culture. In another example, detection of nutrients from a cell culture liquid, such as a supernatant, may provide an indication of cell culture health as a healthy culture will consume the nutrients over time. In yet another example, changes in the health of a cell culture may be tracked using machine learning to interpret spectrometry results.

By collecting microfluidic samples of cell culture liquid of about 1 μL for subsequent spectroscopic analysis, such as surface-enhanced Raman spectroscopy or surface-enhanced Infrared absorption spectroscopy, the cell culture will not be disturbed. Therefore, multiple measurements may be made in a well over a period of time in a single well to measure the change in cell health over the period of time. This microfluidic and nanofluidic platform may be used to manipulate and sample small amounts of cell culture liquid that may include biomarkers from red blood cells, white blood cells, platelets, cancer cells, bacteria, yeast, microorganisms, proteins, DNA, etc. In other examples, the apparatus may be used on other applications than cell cultures. For example, the apparatus may be used to collect microfluidic samples from colloids, and mixtures of inert particles.

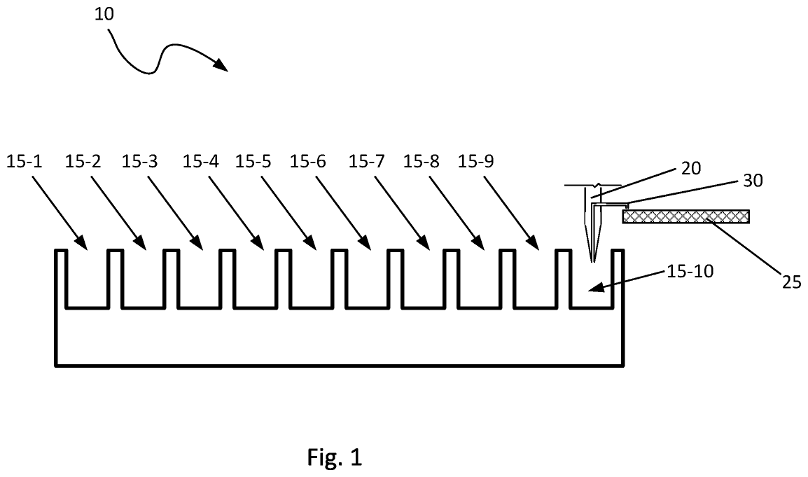

Referring to FIG. 1, an apparatus to collect a microfluidic sample from a plurality of wells is shown at 10. The apparatus 10 includes a plurality of wells 15-1, 15-2, 15-3, 15-4, 15-5, 15-6, 15-7, 15-8, 15-9, and 15-10 (generically, these wells are referred to herein as “well 15” and collectively they are referred to as “wells 15”, this nomenclature is used elsewhere in this description) in which samples may be stored. Furthermore, the apparatus 10 includes a collector 20, a sensor substrate 25, and a microfluid channel 30.

The plurality of wells 15 are to receive samples for which a measurement is to be made. In the present example, the wells 15 are to receive a mixture including liquid. For example, wells 15 may receive a cell culture with a cell culture liquid. In other examples, the wells 15 may receive a homogenous mixture of cells in a solution. In further examples, the wells 15 may include other components in the wells 15 such as reagents for a chemical reaction, chemotherapy drugs and/or unique nutrient mixtures for cells.

In this example, the wells 15 are arranged in a regular array. The dimensions and arrangement are not particularly limited. For example, in the wells 15 may be each about 15 mm in diameter and capable of holding about 3 mL of volume each. Other examples the wells 15 may be larger or smaller. Although the present example shows ten substantially similar wells 15 along a line, it is to be appreciated that the plurality of wells 15 is not limited. For example, other examples may have more or less than ten wells 15. Furthermore, the wells 15 may be arranged in a two-dimensional pattern.

Furthermore, the wells 15 may be another shape such as square, rectangular, or hexagonal. The wells 15 also may be different shapes and sizes within the same array. In such as example, the array of wells 15 may be used to carry out multiple unrelated experiments on a single machine that may be shared among different research groups.

In the present example, the collector 20 is to contact the cell culture liquid in a well 15. After contacting the cell culture liquid, the collector 20 is to draw a microfluidic sample of the cell culture liquid from the well 15. It is to be appreciated in other examples the collector 20 may be used to collect a microfluidic sample of the mixture, which may include cells or other non-liquid components. In further examples not involving cells, the collector 20 may be used to draw microfluidic samples of reagents and/or products in a chemical reaction.

Although the present example shows the collector 20 as having a tip to be inserted into a well 15 from above, it is to be appreciated that the collector is not particularly limited. For examples in other examples, the collector may be a nozzle disposed on the side of the well or at the bottom of the well 15 to dispense a microfluidic sample. Accordingly, the collector 20 may be an integrated component of the well 15.

By removing a microfluidic sample from a well 15 for testing, the overall conditions within the well 15 is not substantially affected. In the present example, this allows the cell culture with a cell culture liquid layer to continue growing under conditions substantially similar to the starting conditions. Accordingly, multiple samples may be taken at predetermined time intervals to measure the cell culture over time to monitor the health and growth of the cell culture within the well 15.

The wells 15 may be used to carry out testing or other experiments on a cell culture and the nature and conditions of the cell culture in each well 15 is not particularly limited. For example, each well 15 may include a culture of cells growing with a cell culture liquid layer in the well 15. The cell culture liquid layer may include various nutrients that allow a cell culture to grow within the well 15. In addition, the cell culture may release compounds, such as various biomarkers into the cell culture liquid. The biomarkers released by the cell culture is not limited and may vary depending on the specific cell culture.

The testing carried out in a well 15 is not particularly limited. In the present example, the well 15 may receive a cell treatment. The cell treatment may include changing a condition such as temperature or chemical composition of the cell culture liquid. In other examples, the cell treatment may be a dose of an antibiotic, drug, or another medical component. The manner by which the cell culture in the well 15 interacts with the medical component is not limited and may involve heating or mixing the cells to increase the rate at which a change to the health of the cell culture occurs. In further examples, the well 15 may receive other components or mixtures, such as chemotherapy drugs, to test the toxicity or inhibition to cell growth. In further examples, the wells 15 may be a direct tissue sample, such as blood or cells from a biopsy to be used for a diagnosis procedure, such as cancer screening.

In the present example, the collector 20 uses capillary action to draw a microfluidic sample from the well 15. Continuing with the example above of collecting cell culture liquid from a cell culture, the collector 20 may be lowered to the surface of the cell culture liquid in a well 15 where capillary action will cause a microfluidic sample to enter the collector 20. In other examples, the collector 20 may use a vacuum to draw a microfluidic sample through a microfluidic channel when the collector 20 is in contact with the content in a well 15.

Furthermore, in some examples, the collector 20 may include a dispensing unit (not shown) to replenish at least some components removed in the microfluidic sample. For example, when the well 15 is to provide a cell treatment to a plurality of cells, the cell treatment may be dissolved or mixed into the cell culture liquid. By removing microfluidic samples, small amounts of cell treatment and other components may also be removed. Accordingly, replenishing components may be used to maintain a consistent condition in the well 15 when large amounts of samples are removed.

The sensor substrate 25 is to receive the microfluidic sample drawn by the collector 20. The sensor substrate 25 is then used to measure a characteristic of the microfluidic sample. Continuing with the present example, the sensor substrate 25 may be to receive a microfluidic amount of the cell culture liquid drawn in by the collector 20. The sensor substrate 25 may then be transferred to an external sensor system for measurement of the characteristic.

The external sensor system may be used to measure a characteristic of the cell culture liquid. In the present example, the sensor system is to measure the characteristic associated with the cells in the well 15 such as biomarkers that may be generated by cells to indicate the health or stress of a cell culture in the well 15. It is to be appreciated that the sensor system is not particularly limited and may be selected based on components of the cell culture liquid that are to be the focus of the measurement. In the present example, the characteristic to be measured may be associated with a cell count or other indication of the health of a sample of the cells in the well 15. This characteristic may be used to determine the effects of a cell treatment, such as an antibiotic or other medical component, added to the well 15. The health of the cell culture may then be used to determine an effective dose of the medical component or a minimum inhibitory concentration of the antibiotic. Therefore, the apparatus 10 may be part of a system to test new drugs to determine effective doses as well 15 as to test antibiotics to determine an antibiotic dose to stop the growth of a bacteria.

In the present example, the external sensor system is not limited and may be any type of sensor capable of measuring a desired characteristic of the microfluidic sample collected by the collector 20. In the present example, the sensor system may be a substrate designed to enhance luminescence from a sample and a spectrometer for detecting signals from a light source to detect spectroscopic signals that may be reflected or transmitted through microfluidic sample. For example, the sensor system may be a Raman spectrometer to carry out surface-enhanced Raman spectroscopy (SERS) after a monochromatic light source, such as a laser, emits light on the microfluidic sample deposited on a SERS substrate. Alternately the microfluidic sample may be mixed with a SERS colloid mixture. This technique may be used to detect the presence of biomarkers produced by healthy cells to provide an indication as to the health of the cells in the well 15 from which the microfluidic sample came. As another example, the sensor system may be an infrared spectrometer to carry out surface-enhanced infrared absorption spectroscopy after depositing the microfluidic sample on a surface-enhanced infrared absorption substrate and exposing it to infrared radiation. This technique may also be used to detect the presence of biomarkers produced by healthy cells to provide an indication as to the health of the cells in the well 15 from which the microfluidic sample came.

In yet another example, the sensor system may be a combination of both a Raman spectrometer and an infrared detector, such that characteristics of the cells may be detected using multiple methods. A single substrate can be used to enhance both Raman scattering and infrared absorption, or a dedicated sensor substrate can be used for each detection method. When using multiple methods, the microfluidic sample may be excited by more than one excitation wavelength of light. In addition, multiple detectors may be used to measure signals from the microfluidic sample.

In the present example, the microfluidic channel 30 is to transport the microfluidic sample from the well 15 via the collector 20 to the sensor substrate 25. For example, the microfluidic channel 30 may be about 10 μm to about 100 μm wide by about 100 μm tall. In other examples, the dimensions of the microfluidic channel 30 may be about 0.5 μm wide by about 10 μm tall. It is to be appreciated that other examples of the microfluidic channel 30 may be about 1.0 μm wide by about 20 μm tall, about 5.0 μm wide by about 50 μm tall, and about 20 μm wide by 500 μm tall. Other examples may involve square shaped microfluidic channels 30 having cross sectional dimensions such as about 0.5 μm by about 0.5 μm, about 1.0 μm by about 1.0 μm, and about 10 μm by about 10 μm. It is to be appreciated that the microfluidic channel 30 may also be replaced with a smaller nanofluidic channel to draw and smaller sample size of cells and solution. In the present example, the microfluidic channel 30 is shown to flow the microfluidic sample from the collector 20 to the sensor substrate 25 using a pump or vacuum system. In other examples, the microfluidic channel 30 may be used to draw the microfluidic sample using capillary action and then moved to the sensor substrate 25 where the microfluidic sample is ejected through the same opening from which the sample was drawn.

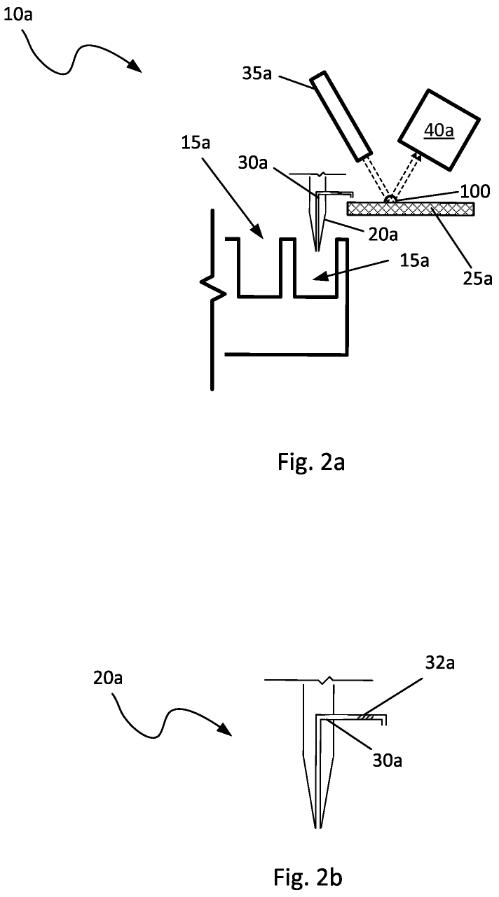

Referring to FIG. 2a and FIG. 2b, another example of an apparatus to collect a microfluidic sample 100 from a plurality of wells 15a is shown at 10a. Like components of the apparatus 10a bear like reference to their counterparts in the apparatus 10, except followed by the suffix “a”. The apparatus 10a includes a plurality of wells 15a, a collector 20a, a sensor substrate 25a, a microfluid channel 30a, and a detector 40a. In some examples, the apparatus 10a may further include a light source 35a to work in combination with the detector 40a.

In the present example, the plurality of wells 15a are to each receive a bacteria culture with a cell culture liquid. In particular, the wells 15a are to initially receive identical mixtures of bacteria culture with cell culture liquid media such that under similar conditions, the bacteria culture in each of the wells 15a is to develop substantially the same over time.

In the present example, the collector 20a collects a microfluidic sample 100 of cell culture liquid from the well 15a. In particular, the collector 20a may be lowered to the surface of the cell culture liquid in the well 15a where capillary action will cause a microfluidic sample 100 to enter the collector 20a. It is to be appreciated that the volume of the microfluidic sample 100 is not limited and may vary between applications. For example, the microfluidic sample 100 in the present example of wells 15b may be about 1 nL. In other examples, the volume of the microfluidic sample 100 may be no more than about 10 pL or as large as about 1 μL. In other examples, the microfluidic sample 100 may be no more than about 100 pL, about 500 pL, about 1 nL, about 5 nL, about 10 nL, about 50 nL, about 100 nL, or about 500 nL. Furthermore, the cell culture liquid of the well 15a may include various components that may be used to evaluate the overall health of the bacteria in the well 15a. For example, the cell culture liquid may include biomarkers produced by healthy bacteria, and nutrients that are consumed by healthy bacteria. By determining the presence and/or concentration of either one of these components, the general health of the bacteria in the well 15a may be estimated.

The sensor substrate 25a is to receive the microfluidic sample 100 drawn by the collector 20a. The sensor substrate 25 is then used to measure a characteristic of the microfluidic sample 100. Continuing with the present example, the sensor substrate 25a may be to receive a microfluidic amount of the cell culture liquid drawn in by the collector 20a. In the present example, the sensor substrate 25a is part of an integrated sensor system for measurement of the characteristic. In the present example, the sensor system includes a light source 35a and a detector 40a.

The detector 40a is to measure a characteristic of the cell culture liquid on the sensor substrate 25a. The detector 40a is not limited and may be any type of detector capable of measuring a desired characteristic of cell culture liquid. In the present example, the detector 40a may be a spectrometer for detecting signals from a light source to detect spectroscopic signals that may be reflected or transmitted through the microfluidic sample 100. For example, the detector 40a may be a Raman spectrometer to carry out surface-enhanced Raman spectroscopy after a monochromatic light source 35a, such as a laser, emits light on the microfluidic sample 100. This technique may be used to detect the presence of biomarkers produced by healthy cells to provide an indication as to the health of the bacteria in the well 15a. As another example, the detector 40a may be an infrared spectrometer to carry out surface-enhanced infrared absorption spectroscopy after exposing the microfluidic sample 100 to infrared radiation. This technique may also be used to detect the presence of biomarkers produced by healthy cells to provide an indication as to the health of the bacteria in the well 15a. In yet another example, the detector 40a may be a combination of both a Raman spectrometer and an infrared detector, such that characteristics of the cells may be detected using multiple methods.

In the present example, the microfluidic channel 30a is to transport the microfluidic sample 100 from the well 15a via the collector 20a to the sensor substrate 25a for measuring a characteristic. In this example, the microfluidic channel 30a further includes a filter 32a to remove large molecules, such as proteins and lipids, which may interfere with the measures carried out by the detector 40a. The filter 32a is not limited and may include polytetrafluoroethylene, cellulose acetate, cellulose nitrate, polypropylene, polyethylene terephthalate, olefinic or nylon membranes. In other examples, a glass filter may also be used to remove proteins.

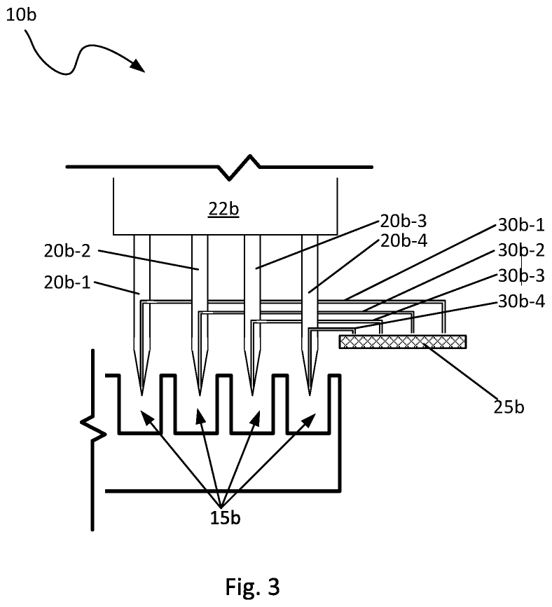

Referring to FIG. 3, another example of an apparatus to collect a microfluidic sample 100 from a plurality of wells is shown at 10b. Like components of the apparatus 10a bear like reference to their counterparts in the apparatus 10, except followed by the suffix “b”. The apparatus 10b includes a plurality of wells 15b, a plurality of collectors 20b-1, 20b-2, 20b-3, and 20b-4 (generically, these collectors are referred to herein as “collector 20b” and collectively they are referred to as “collectors 20b”), a sensor substrate 25b, a plurality of microfluid channels 30b-1, 30b-2, 30b-3, and 30b-4 (generically, these microfluid channels are referred to herein as “microfluidic channel 30b” and collectively they are referred to as “microfluidic channels 30b”), and a multi-tip sampler 22b.

In the present example, the collectors 20b are to draw a plurality of microfluidic samples from the plurality of wells 15b. In particular, the present example may provide the capability of drawing the plurality of microfluidic samples simultaneously to reduce sampling time and increase the speed at which the wells 15b may be tested.

In the present example, each of the collectors 20b include an associated microfluidic channel 30b which may be used to transport microfluidic samples to the sensor substrate 25b. The sensor substrate is to receive a plurality of microfluidic samples, such as microfluidic cell culture liquid samples from the wells 15b. It is to be appreciated that the microfluidic samples may be dispensed onto known locations of the sensor substrate 25b to provide for rapid measurement of multiple microfluidic samples on the substrate. For example, the sensor substrate 25b may be moveable and aligned at multiple positions within a spectrometer to provide rapid measurements without full removal and installation of a sensor substrate 25b between each sample.

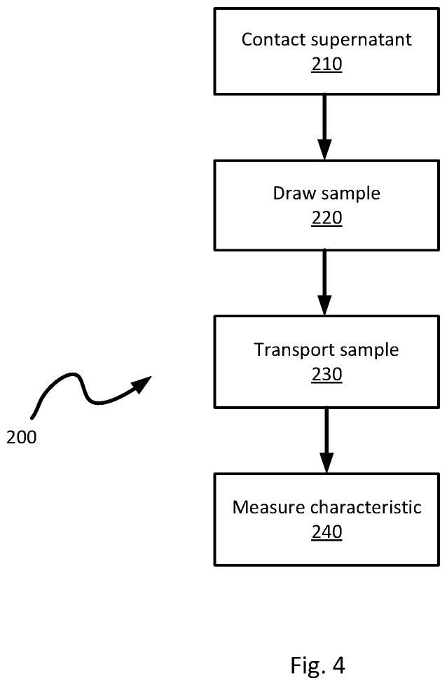

Referring to FIG. 4, a flowchart of a method of collecting a microfluidic sample from a plurality of wells is shown at 200. In order to assist in the explanation of method 200, it will be assumed that method 200 may be performed with any of the apparatus 10, 10a, or 10b described above. Indeed, the method 200 may be one way in which apparatus 10, 10a, or 10b may be configured to collect a microfluidic sample from a plurality of wells for subsequent measurement of a characteristic. Furthermore, the following discussion of method 200 may lead to a further understanding of the apparatus 10, 10a, or 10b and their various components. For purposes of the following discussion, it is to be assumed that the method 200 is carried out on the apparatus 10. Furthermore, it is to be emphasized, that method 200 may not be performed in the exact sequence as shown, and various blocks may be performed in parallel rather than in sequence, or in a different sequence altogether.

Beginning at block 210, the collector 20 is lowered to contact a cell culture liquid in a well 15. The manner by which the collector 20 is lowered is not limited. For example, the collector 20 may be mounted on moveable tracks to position the collector above a well 15 from which a microfluidic sample of cell culture liquid is to be collected. In the present example, the collector 20 contacts the surface of the cell culture liquid and stops.

It is to be appreciated that the collector 20 or the motion system may include additional sensors to detect contact with the surface of the cell culture liquid. The collector is stopped at the surface of the cell culture liquid to avoid agitating the cell culture in the well 15. In particular, the collector 20 may be moved such that the cell culture at the bottom of the well 15 is not substantially disturbed to reduce the risk of collecting cells via the collector 20.

In the present example, the well 15 may receive a cell treatment prior to the first contact of the collector 20 on the cell culture liquid. The cell treatment may include changing a condition such as temperature or chemical composition of the cell culture liquid. In other examples, the cell treatment may be a dose of an antibiotic, drug, or another medical component. The manner by which the cell culture in the well 15 interacts with the medical component is not limited and may involve heating or mixing the cells to increase the rate at which a change to the health of the cell culture occurs.

Block 220 involves drawing a microfluidic sample of cell culture liquid from the well 15. It is to be appreciated that by drawing a microfluidic sample, the condition in the well 15 is maintained to be substantially the same. Accordingly, taking microfluidic samples allows the cell culture with a cell culture liquid layer to continue growing under conditions substantially similar to the starting conditions. This provides the feature of taking multiple samples at predetermined time intervals from the same well 15 to measure changes in the cell culture over time to monitor the health and growth of the cell culture within the well 15.

Block 230 transports the microfluidic sample to the sensor substrate 25. The manner by which microfluidic sample is transported is not limited and may involve the microfluidic channel 30 being used to transport the microfluidic sample from the well 15 via the collector 20 to the sensor substrate 25. In other examples, it is to be appreciated that the microfluidic channel 30 may be replaced with a nanofluidic channel to draw and smaller sample size of cells and solution. In the present example, the microfluidic channel 30 may flow the microfluidic sample from the collector 20 to the sensor substrate 25. In some examples, the microfluidic sample may be passed through a filter, such as the filter 32a, to remove large molecules prior to placing the microfluidic sample on the sensor substrate 25. In other examples, the microfluidic channel 30 may be used to draw the microfluidic sample using capillary action and then the whole collector 20, or microfluidic channel may be moved to the sensor substrate 25 where the microfluidic sample is ejected through the same opening from which the sample was drawn.

Next, block 240 involves measuring a characteristic of the cell culture based on the microfluidic sample on the sensor substrate 25. In the present example, the characteristic to be measured may be an indicator of cell culture health in a well 15. In this example, the characteristic may be used to determine the effectiveness of a treatment. For example, if the cell culture is a bacterial culture treated with an antibiotic, the characteristic may be a signal from a spectroscopy technique associated with a biomarker generated by the bacteria. Accordingly, if the intensity of the signal is weak, it may provide an indication that the cell culture is in poor health. Conversely, if the intensity of the signal is strong, it may provide an indication that the cell culture remains healthy despite the antibiotic treatment. The measurements are not particularly limited. For example, the measurements may involve performing surface-enhanced Raman spectroscopy on the bacteria to look for biomarkers or performing surface-enhanced infrared spectroscopy on the bacteria to look for biomarkers.

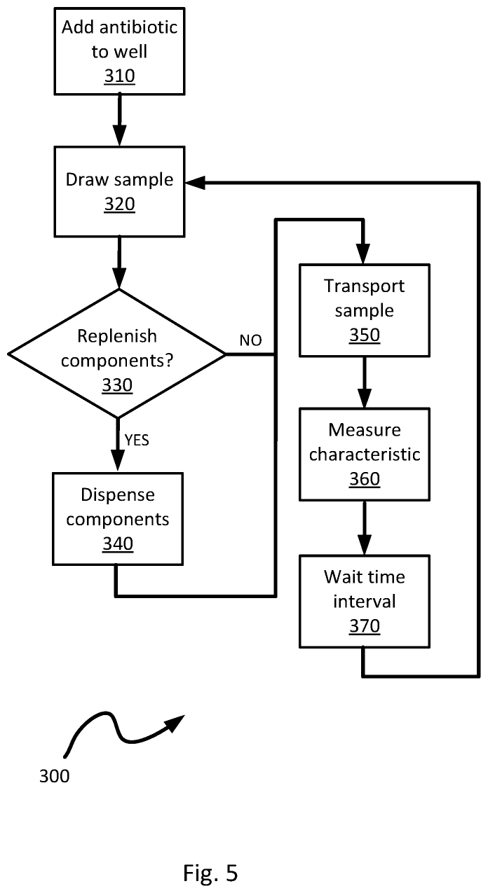

Referring to FIG. 5, a flowchart of a method of using microfluidic samples from a plurality of wells to determine a minimum inhibitory concentration of an antibiotic is shown at 300. In order to assist in the explanation of method 300, it will be assumed that method 300 may be performed with any of the apparatus 10, 10a, or 10b described above. Furthermore, the following discussion of method 300 may lead to a further understanding of the apparatus 10, 10a, or 10b and their various components. For purposes of the following discussion, it is to be assumed that the method 300 is carried out on the apparatus 10. Furthermore, it is to be emphasized, that method 300 may not be performed in the exact sequence as shown, and various blocks may be performed in parallel rather than in sequence, or in a different sequence altogether.

Beginning at block 310, a dosage of antibiotic is added to a well 15 containing a bacteria culture. In the present example, the apparatus 10 includes a plurality of wells 15, which are to each receive a bacteria culture with a cell culture liquid. In particular, the wells 15 are to initially receive substantially identical mixtures of bacteria culture with and cell culture liquid such that under similar conditions, the bacteria culture in each of the wells 15 is to develop substantially the same over time. At the beginning of the testing for the minimum inhibitory concentration of an antibiotic, different antibiotic doses are added to each well 15.

At block 310, the collector 20 is lowered to contact a cell culture liquid in a well 15 to draw a sample of the cell culture liquid. It is to be appreciated that by drawing a microfluidic sample, the condition in the well 15 is maintained to be substantially the same. Accordingly, taking microfluidic samples allows the bacteria culture with a cell culture liquid layer to continue growing under conditions substantially similar to the starting conditions. This provides the feature of taking multiple samples at predetermined time intervals from the same well 15 to measure changes in the bacteria culture over time to monitor the health and growth of the bacteria culture within the well 15.

Block 330 involves determining whether components in the well 15 are to be replenished. Although microfluidic samples are drawn in block 320, it is to be appreciated that after multiple samples are drawn, some components such as nutrients and antibiotics may be removed from the well. Accordingly, to maintain the same dosage, such components may be added into the well 15 from time to time. The determination of when components is to be replenished in a well 15 is not particularly limited. For example, the components may be replenished after a predetermined number of sample collections. In other examples, measurement may be taken to determine the amount of each component removed in the microfluidic sample. This may be an additional measurement step in some examples. In other examples, the measurements may be made at block 360 along with the measurements for the characteristic of the microfluidic sample. If it is determined that the components of the well 15 are to be replenished, the method 300 proceeds to block 340. If it is determined that the components of the well 15 are not to be replenished, the method proceeds directly to block 350.

Block 340 involves replenishing components in the well 15. The manner by which the components are replenished is not particularly limited. In the present example, the collector 20 may include a dispensing unit (not shown) to replenish components removed by drawing microfluidic samples over time.

Block 350 transports the microfluidic sample to the sensor substrate 25. The manner by which microfluidic sample is transported is not limited and may involve the microfluidic channel 30 being used to transport the microfluidic sample from the well 15 via the collector 20 to the sensor substrate 25. In the present example, the microfluidic channel 30 may flow the microfluidic sample from the collector 20 to the sensor substrate 25. In some examples, the microfluidic sample may be passed through a filter, such as the filter 32a, to remove large molecules prior to placing the microfluidic sample on the sensor substrate 25. In other examples, the microfluidic channel 30 may be used to draw the microfluidic sample using capillary action and then the whole collector 20, or microfluidic channel may be moved to the sensor substrate 25 where the microfluidic sample is ejected through the same opening from which the sample was drawn.

Next, block 360 involves measuring a characteristic of the bacteria culture based on the microfluidic sample on the sensor substrate 25. In the present example, the characteristic to be measured may be an indicator of bacteria culture health in a well 15. In this example, the characteristic may be used to determine the effectiveness of a treatment. For example, the characteristic may be a signal from a spectroscopy technique associated with a biomarker generated by the bacteria. Accordingly, if the intensity of the signal is weak, it may provide an indication that the bacteria culture is in poor health. Conversely, if the intensity of the signal is strong, it may provide an indication that the bacteria culture remains healthy despite the antibiotic treatment. The measurements are not particularly limited. For example, the measurements may involve performing surface-enhanced Raman spectroscopy on the bacteria to look for biomarkers or performing surface-enhanced infrared absorption spectroscopy on the bacteria to look for biomarkers

Block 370 waits a predetermined period of time before looping the method back to block 320. It is to be appreciated that block 370 provides for periodic measurements of the bacteria culture. This may provide an indication of the minimum inhibitory concentration of an antibiotic by observing the health of bacteria cultures over time. The length of time block 370 delays is not particularly limited and may depend on the bacteria and antibiotic. For example, taking frequent measurements may affect the adversely conditions in the well 15 such the that results for the minimum inhibitory concentration of an antibiotic may be unreliable. However, too infrequent measurements may result in missing data or a delayed answer. In the present example, the period of time between measurements may be about 15 minutes to about 60 minutes. In other examples, the period of time may be shorter for faster growing cell cultures or longer for slower growing cell cultures.

It should be recognized that features and aspects of the various examples provided above may be combined into further examples that also fall within the scope of the present disclosure.

What is claimed is:

1. An apparatus comprising:
a plurality of wells, wherein each well is to receive a cell culture and a cell culture liquid;
a collector to contact the cell culture liquid in a well selected from the plurality of wells, the collector to draw a microfluidic sample of the cell culture liquid from the well;
a sensor substrate to receive the microfluidic sample, wherein the sensor substrate is to be used to measure a characteristic of the cell culture liquid; and
a microfluidic channel to transport the microfluidic sample from the collector to the sensor substrate.

2. The apparatus of claim 1, wherein each well in the plurality of wells is to receive a cell treatment.

3. The apparatus of claim 2, wherein the cell treatment is an antibiotic.

4. The apparatus of claim 3, further comprising a detector to measure the characteristic of the cell culture liquid on the sensor substrate.

5. The apparatus of claim 4, wherein the detector is a spectrometer to measure the characteristic.

6. The apparatus of claim 5, wherein the characteristic is associated with a biomarker to indicate cell health in the well.

7. The apparatus of claim 1, further comprising additional collectors to draw a plurality of microfluidic samples from the plurality of wells.

8. The apparatus of claim 7, wherein the collector and the additional collectors draw the plurality of microfluidic samples simultaneously.

9. A method comprising:
contacting a cell culture liquid in a well with a collector;
drawing a microfluidic sample of the cell culture liquid, wherein drawing the microfluidic sample maintains a condition in the well;
transporting the microfluidic sample to a sensor substrate; and
measuring a characteristic of the microfluidic sample on the sensor substrate.

10. The method of claim 9, further comprising replenishing components of the microfluidic sample into the well.

11. The method of claim 9, further comprising receiving a cell treatment in the well.

12. The method of claim 11, wherein the cell treatment is an antibiotic.

13. The method of claim 12, wherein the characteristic is associated with a biomarker to indicate cell health in the well.

14. An apparatus comprising:
a plurality of wells, wherein each well is to receive a bacteria culture and a cell culture liquid;
a multi-tip sampler to draw a plurality of microfluidic cell culture liquid samples from the plurality of wells; and
a sensor substrate to receive the plurality of microfluidic cell culture liquid samples, wherein the sensor substrate is moveable to a spectrometer to detect biomarkers in a cell culture liquid sample selected from the plurality of microfluidic cell culture liquid samples, wherein the biomarkers provide an indication of bacteria health.

15. The apparatus of claim 14, wherein the spectrometer determines the bacteria health to determine whether an antibiotic dose associated with a well in the plurality of wells provides a minimum inhibitory concentration.
